(19) United States
(12) Patent Application Publication
(10) Pub. No.: US 2016/0361252 A1
(43) Pub. Date: Dec. 15, 2016

(54) ANHYDROUS MULTIPHASE GEL SYSTEM

(71) Applicant: Patrick Franke, Berlin (DE)

(72) Inventor: Patrick Franke, Berlin (DE)

(21) Appl. No.: 15/185,210

(22) Filed: Jun. 17, 2016

Related U.S. Application Data
(63) Continuation of application No. 14/031,619, filed on Sep. 19, 2013, now abandoned.
Application No. 14/031,619 is a continuation of application No. 11/989,968, filed on Mar. 8, 2010, now abandoned.

(30) Foreign Application Priority Data
Aug. 4, 2005 (DE) 102005037844.7

(51) Int. Cl. A61K 9/00; A61K 31/436; A61K 47/34

(52) U.S. Cl. CPC A61K 9/0014; A61K 47/34; A61K 31/436

(57) ABSTRACT

An anhydrous multiphase gel system consisting of an outer lipid matrix and an inner phase gelled by means of a polymer is described, which can be obtained by a) Melting the lipid phase with the formation of a liquid lipid phase, b) Mixing and homogenizing polymers or polymer blends capable of swelling with the formation of a polymer phase to be dispersed, c) Combining the polymer phase with the liquid lipid phase and homogenizing the phases, and d) Cold stirring the phase mixture until a solid gel-like mixed structure of the entire system is formed. The anhydrous multiphase gel system is particularly suitable for taking up difficultly soluble active substances in high concentration and for providing topical and transdermal applications. The described system is called an EDRS, “Entrapped Drug Reservoir System”.

The present invention relates to a topically applicable composition in the form of an anhydrous multiphase gel system.

A very wide variety of topically applicable compositions are known in the prior art. Two representatives of these systems for dermal application are creams and salves.

Creams are emulsions that comprise a dispersed phase and a dispersing agent. Here, one distinguishes essentially between water-in-oil and oil-in-water emulsions, depending on which type of emulsifiers are used. In each case, however, systems containing water are involved.

Many users of creams find it particularly advantageous that these creams are easy to distribute and do not feel sticky and greasy on the skin.

A disadvantage of creams, however, is that they possess only slight occlusion. A hydrating of the stratum corneum of the skin is achieved by means of occlusion and, in this state, the stratum corneum can absorb three to five times its weight in water and is permeable.

In addition, the application of creams as a vehicle for active substances is limited, since the water that is necessarily present in lotions very greatly limits or even excludes the incorporation of hydrolysis-sensitive substances.

In contrast, salves are usually semi-solid preparations based on lipid material, which are suitable for external application. One type of salve is anhydrous hydrocarbon salves (lipid salves), which contain linear or branched hydrocarbons with chain lengths of C₁₆ to C₃₀ and may also include cyclic alkanes. A typical formulation contains liquid hydrocarbons (mineral oils and liquid paraffins) mixed with hydrocarbons with longer alkyl chains (mean chain length of approximately 35 to 50 carbon atoms, usually n- and iso-paraffins) with high melting points, for example, vaseline, hard paraffins and waxes.

It is advantageous that these anhydrous compositions have high occlusion on the skin.

It is a disadvantage, however, that lipid salves for the most part are very sticky and greasy and thus induce an unpleasant sensation for many users when they are used. In addition, they can only be poorly distributed on the skin.

Also, the applicability of these anhydrous salves as slow-release systems for active substances that are to be administered topically are limited, since many active substances possess a relatively poor solubility in a hydrocarbon salve. Therefore, the incorporation of these substances in such topically applicable systems is very limited and frequently it is not possible to incorporate them in an effective concentration.

The solvent volume available for an active substance in the hydrocarbon salve can be increased to a certain extent by processing the basic salve substances with solvents that are miscible with hydrocarbons, such as, for example, isopropyl myristate. In this way, of course, only the solubility of active substances that are soluble in solvents miscible with hydrocarbons is increased in the lipid salve.

In order to also incorporate active substances which are insoluble or soluble to only a very limited extent in solvents miscible with hydrocarbons in the above-described hydrocarbon salves, solvents that are immiscible with hydrocarbons have also been introduced into hydrocarbon salves. Of course, previously, these solvents could only be introduced in an amount up to 5% in the classical lipid salves. Otherwise, the preparation is unstable (syneresis); i.e., there occurs a “sweating out” of the solvent.

Another essential aspect which determines the quality of a topically applicable composition is that it effects a high penetration into the skin of the cosmetic, personal care and/or therapeutic components contained in it.

It is known that the penetration can be increased by the incorporation of penetration promoters (enhancers). Thus substances that solvatize the polar components of skin lipids, for example, water, dimethyl sulfoxide (DMSO) and ethanol, act to promote penetration both for hydrophilic as well as lipophilic active substances, due to the resulting increase in the volume of the lipid layers of the skin. Substances that interact with the non-polar components of skin lipids can influence the micofluidity of the membranes and thus also improve the penetration. These include isopropyl myristate, isopropyl palmitate and oleic acid. Higher alcohols such as propylene glycol, glycerol and sorbitol can be incorporated directly into the aqueous layers between the lipid double membranes and here improve the solubility of many active substances and this frequently also increases the penetration.

Therefore, many different effects of the vehicle on the stratum corneum are possible and these effects can influence the permeation of an active substance through the skin. In most cases, this leads to an acceleration in penetration, which is predominantly explained by the following mechanisms: Most frequently, an interaction between the vehicle and the intercellular lipids occurs in the stratum corneum, which can lead either to a fluidizing of the lipid or to a dissolving out of several lipid fractions from the stratum corneum. Interactions with lipids, with the polar head groups and/or with the lipophilic fatty acid groups of the lipids, are possible, each time depending on the property of the vehicle. In addition, the penetration of relatively large quantities of vehicle into the barrier leads to a co-solvent effect. The assumption is that vehicle components can penetrate into the skin and in this way entrain the active substance.

In addition to the penetration of vehicle, the reinforced hydrating of the stratum corneum under occlusive conditions should also be considered, and as described above, the hydrocarbon salves known in the prior art show a strong occlusion. The acceleration of penetration in all cases occurs due to an increase in the diffusion coefficient of the active substance in the stratum corneum and/or due to an increase of the saturation concentration of the active substance in the barrier.

If an active substance only has a small tendency to pass from the topically applicable composition into the skin, however, the composition must be altered such that an optimal dermal availability is achieved.

The object of the present invention is thus to provide a topically applicable composition, which possesses the advantageous occlusion properties of a lipid salve, and also shows application properties similar to a cream or a lotion, and additionally can function as a vehicle with a high penetration capacity for a plurality of active substances, even those that are sensitive to hydrolysis. The inventors name the system according to the invention by the acronym “EDRS” or “EDR system”, as the abbreviation for “Entrapped Drug Reservoir System”.

According to the invention, the object is achieved by providing an anhydrous multiphase gel system consisting of an outer lipid matrix and an inner phase gelled by means of a polymer that can be obtained by

a) Melting the lipid phase with the formation of a liquid lipid phase,
b) Mixing and homogenizing polymers or polymer blends capable of swelling with the formation of a polymer phase to be dispersed,
c) Combining the polymer phase with the liquid lipid phase and homogenizing the phases, and
d) Cold stirring the phase mixture until a solid gel-like mixed structure of the entire system (EDRS) is formed.

According to the invention, a system is preferred, wherein the lipid phase contains lipids that are compatible with the skin.

It is particularly preferred that the lipids are selected from petrolatum, paraffin, microcrystalline wax, squalene, cetylstearyl octanoate, ethyl oleate, glyceryl tricaprylate/caprate, myristyl myristate, propylene glycol dicaprate, cetyl esters, isopropyl myristate, isopropyl palmitate, mono-, di- and triglycerides, ethoxylated glycerides, polyethylene glycol esters, sorbitan esters, solid lipids, e.g., Novata™, dibutyl adipate, ethyl linoleate, crodamols, such as ethylhexyl cocoate and others, isocetyl stearate, Cetiol™, cetyl palmitates, e.g. Cutina CP™, cetyl alcohol, oleyl alcohol, stearyl alcohol, dicaprylyl ether, oleic acid, waxes, such as jojoba wax and beeswax, cholesterins, polyethylene glycols, lanolin, lanolin alcohols, silicone oils and their mixtures.

Further, a system is preferred according to the invention, wherein the polymers are cellulose derivatives, acrylate polymers and their derivatives or their mixture.

It is also particularly preferred that the cellulose derivatives are hydroxypropylcellulose, carboxymethylcellulose, methylcellulose, hydroxypropylmethylcellulose, hydroxyethylcellulose and their derivatives or their mixtures.

It is also particularly preferred that the acrylate polymers involve crosslinked acrylate polymers.

A system is also preferred wherein the polymers or polymer mixtures that can be swollen are swollen by means of swelling agents containing OH groups.

It is thus preferred that the swelling agent is a monohydric to trihydric aliphatic alcohol with a chain length of up to 5 carbon atoms or mixtures thereof.

It is also most particularly preferred that the monohydric aliphatic alcohols are selected from ethanol, n-propanol and isopropanol or mixture thereof.

In addition, it is preferred according to the invention that the polyols are selected from glycerin, propylene glycol and 1,2-pentanediol or mixture thereof.

It is also preferred that the swelling agent comprises, in addition, carbonic acid diesters or mixtures of carbonic acid diesters.

It is particularly preferred here that the carbonic acid diesters are selected from the group: ethylene carbonate, propylene carbonate and other homologs of ethylene carbonate and mixture thereof.

It is further preferred that the swelling agent additionally comprises diethylene glycol monoethyl ether, polyoxylated capryl/capric acid glycerides, dimethyl isosorbide and/or additional pharmaceutically compatible solvents or mixtures thereof.

A system according to the invention is most particularly preferred, wherein the lipid phase and/or the polymer phase contain(s) active components.

It is also preferred that the lipid phase and the polymer phase contain different active components.

It is particularly preferred that the active components are selected from skin-care substances, skin-coloring substances, UV protectors, pharmaceutically active substances or mixtures thereof.

Preferred active components are selected, for example, from polidocanol, synthetic tanning substances, antiseptics such as chlorhexidine and triclosan, antibiotics such as fusidic acid, erythromycin, tetracycline, clindamycin, peptide antibiotics, antimycotics such as imidazole derivatives, terbenafine, ciclopirox, salicylic acid, zinc pyrithione, topical corticosteroids such as, e.g., methylprednisolone aceponate, clobetasol, mometasone fuorate, topical macrolides such as tacrolimus and pimecrolimus, oligonucleotides for gene therapy such as si-RNA and ribozymes, antihistamines, immunsuppressants such as cyclosporin, azathioprine and mycophenolate mofetil, anthralines such as cignolin and dithranol, vitamin D3 analogs such as calcipotriol, topical retinoids, urea, lactic acid, fumaric acid ester, azelaic acid, hydroquinone, benzoyl peroxide, non-steroidal antiphlogistics, sex hormones such as estrogens and androgens, cytostatics, UV protectors such as stilbene derivatives, make-up (camouflage), plant extracts such as green tea extract, Centella asiatica extract, willow bark extract, birch extract, green tea extract, tea olive oil, olive leaf extract, Aloe vera extract, marigold extract, passion flower extract, Hamamelis extract, chamomile extract, tea olive oil*, bearberry leaf extract and licorice root extract, for example as 18β-glycyrhetinic acid (Zn combination), fruit acids such as α-hydroxy acids, β-hydroxy acids and polyhydroxy acids (PHA), malic acid, malonic acid, citric acid, or mixtures thereof. The enumeration of active substances that can be used is not conclusive. Of course, other active substances may also be introduced into the system according to the invention.

It is particularly preferred that the active component is introduced together with a solubilizing agent.

A preferred system according to the invention additionally comprises one or more additives useful for a topically applicable composition.

Another subject of the present invention is the use of a system according to the invention for the preparation of a pharmaceutical composition for application onto the skin, the mucous membranes and/or on wound surfaces.

It is used preferably for the preparation of a product for human or veterinary medicine, thus for application in humans and in animals.

Another subject of the present invention is a method for the production of an anhydrous multiphase gel system consisting of an outer lipid matrix and an inner phase gelled by means of a polymer, wherein

a) The lipid phase is melted with the formation of a liquid lipid phase,
b) Polymers or polymer blends capable of swelling are mixed and homogenized with the formation of a polymer phase to be dispersed,
c) The polymer phase and the liquid lipid phase are combined and the phases are homogenized, and
d) The phase mixture is cold stirred until a solid, gel-type mixed structure of the system is formed.

A method is preferred, wherein an active substance is added to the polymer phase, the lipid phase or both phases.

It has been found surprisingly that the compositions according to the invention possess pronounced advantageous properties. They have an occlusion that is comparable to that of a lipid salve and additionally possess the positive application properties of a cream, such as a pleasant feel on the skin for the user and they are easy to distribute on the skin.

In addition, it has been shown that the compositions according to the invention are particularly suitable as vehicles that can be topically applied for the most varied substance classes.

The compositions according to the invention permit the incorporation of the most varied substance classes in effective quantities. The active substance can be taken up either in the outer lipid matrix or in the gelled dispersed phase, which provides a solution reservoir. And, in addition, different active substances can be contained in a composition according to the invention by introducing them together into one phase or distributing them to the two phases of the system.

And also the penetration of the cosmetic, personal-care and/or therapeutic substances contained in the composition according to the invention into the skin has proven to be very advantageous.

These surprising properties of the compositions according to the invention obviously result from the fact that a stable, multiphase gel system is formed.

The advantage of such a compartmentation in the form of a coherent lipid phase and a gelled, dispersed phase enclosed therein, which forms a reservoir, has been shown, for example, by the fact that the composition according to the invention is excellently suitable, among other things, as a solution vehicle for hydrophobic active substances, which are only slightly soluble or practically insoluble in effective concentrations in hydrocarbons, or must not come into contact with water due to their sensitivity to hydrolysis (e.g., TIMS (e.g. tacrolimus, pimecrolimus, ascrolimus), corticoids and hormones, antibiotics). This was demonstrated by means of an induced contact dermatitis in mice.

It is particularly advantageous that the composition according to the invention permits the stable incorporation of non-aqueous solvents such as, e.g., propylene carbonate, propylene glycol, glycerin, of concentrations up to 30% in the system, and the uptake of ethanol and other short-chain alcohols of up to 25%.

Therefore, an essentially greater solvent reservoir is available for active substances, compared with topically applicable systems with comparable application properties known in the prior art.

An essential advantage of the multiphase gel composition according to the invention thus lies in the effective solubilizing of difficultly soluble active substances in effective concentrations, which is not possible for the most part in classical hydrocarbon salves.

And it has also been shown that the special properties of the system according to the invention improve the dermal introduction of active substances that have only a small tendency to penetrate from a composition into the skin with the use of solubilizers or solvent mixtures, in comparison to systems known in the prior art.

Of course, according to the known prior art, the use of solubilizers or solvent mixtures that increase the saturation solubility of an active substance in the formulation in the creams and hydrocarbon salves known in the prior art is limited by the fact that only limited quantities of penetration enhancers can be incorporated into the systems without dramatically reducing the stability of the system.

This property was particularly observed when the solubilizer and the active substance were introduced into the inner phase of the system, which is present as a discrete compartment in the composition according to the invention.

In contrast, in the system according to the invention, an individual solvent compartment is produced. Penetration enhancer and active substance are present herein in high concentrations and close spatial proximity, which leads to a better solubility of the active substance in the system and consequently also to a better penetration through the skin.

It proceeds from here that the concentration gradient of the active substance increases, while the rate of penetration also increases simultaneously. Therefore, in the system according to the invention, by the addition of the penetration enhancer, there is achieved an increased concentration of active substance in the deeper layers of the skin, since these substances in turn diffuse into the skin and reduce the barrier function of the stratum corneum.

The increase in penetration is possible due to the selection of the type and quantity of the solvent, as a function of the active substances incorporated and the selection of the concentration and the site of loading the system.

Thus, in systems according to the invention, both of the essential aspects for the improvement of penetration of active substances into the skin are achieved, i.e., occlusion effect and penetration enhancement, due to compatible solvents that are accessible to the skin and promote the incorporation of active substances into the skin.

In the sense of this invention, a multiphase gel system denotes a system which is formed of two or more phases.

In connection with the present invention, the term “active substance” refers to a substance which exercises a desired cosmetic, personal-care and/or therapeutic effect directly on the skin or on the organism of the user overall.

In accordance with the invention, the term “active concentration” refers to the concentration which is necessary in order to provide the desired cosmetic, personal-care and/or therapeutic effect.

The use of the term “cosmetic effect” in comparison to “personal-care effect” will emphasize that the compositions according to the invention not only can physiologically improve the state of the skin, but can also be used in the field of decorative cosmetics. An example of this, in addition to the decorative covering of the skin as makeup, is also the covering of skin sites such as birthmarks, scars and skin disfigurements as often occur as a consequence of diseases, such as AIDS, for example, as so-called “camouflage”.

Anhydrous in the sense of the present invention means that up to 1% water can be contained in the composition. Anhydrous solvents according to the invention may contain up to 5% water. Thus, for example, it has been shown that ethanol for use according to the invention may contain up to 4.5% water (azeotrope).

Polymers that can be swollen by means of OH groups can be selected from acrylate polymers or blends thereof.

As examples, let the following several products of the company Noveon Inc. be named: Carbopol 934 NF, Carbopol 934P NF, Carbopol 940 NF, Carbopol 971P NF, Carbopol 71G NF, Carbopol 974P NF, Carbopol 980 NF, Carbopol 981 NF, Carbopol 1342 NF, Carbopol 5984 EP, Pemulen TR-1 NF, Pemulen TR-2 NF, Noveon AA-1 USP, Noveon CA-1 USP and Noveon CA-2 USP.

In connection with the present invention, the expression “short-chain alcohol” refers to a monohydric to trihydric aliphatic alcohol with up to five carbon atoms.

All of the method steps for the production of the compositions according to the invention can be produced by techniques that are known to a person of average skill in the art. Thus, the mixing and homogenizing of the components in step b) for the production of the dispersed polymer phase are produced by currently used stirring systems and homogenizers.

The viscosity of the produced polymer phase can be adjusted by heating, for example.

In addition to the already listed components, the composition according to the invention may further comprise one or more additives useful for a topically applicable composition. For example, these additives may be selected from dyes, aromatic substances, preservatives and absorption-promoting agents.

Embodiment examples of the invention will be presented below as well as results of investigations which show the properties and advantages of the compositions according to the invention, also in comparison with conventional topically applicable systems.

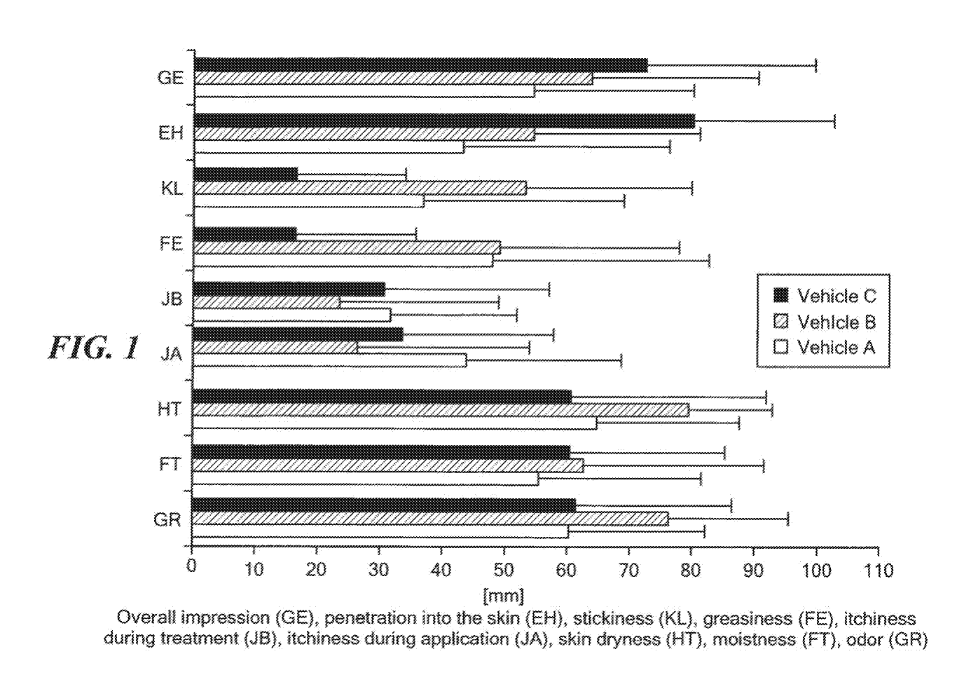

The experimental results will be explained on the basis of FIGS. 1 to 5.

Herein is shown:

FIG. 1

Results of an application study on human skin (measured value: skin feeling in general) according to Example 7

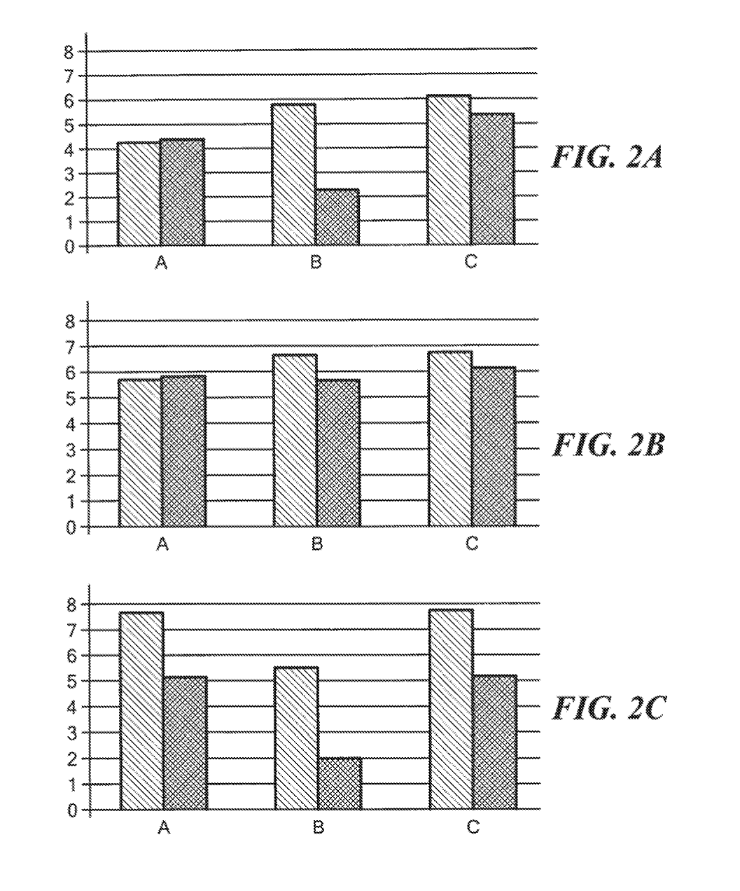

FIGS. 2a-2c

Results of an application study on human skin (measured value: effect on lesions) according to Example 7

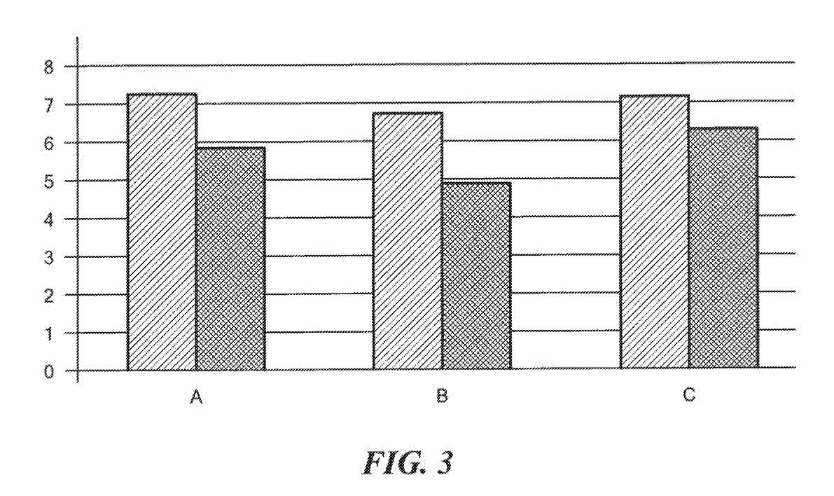

FIG. 3

Results of an application study on human skin (measured value: total effect) according to Example 7

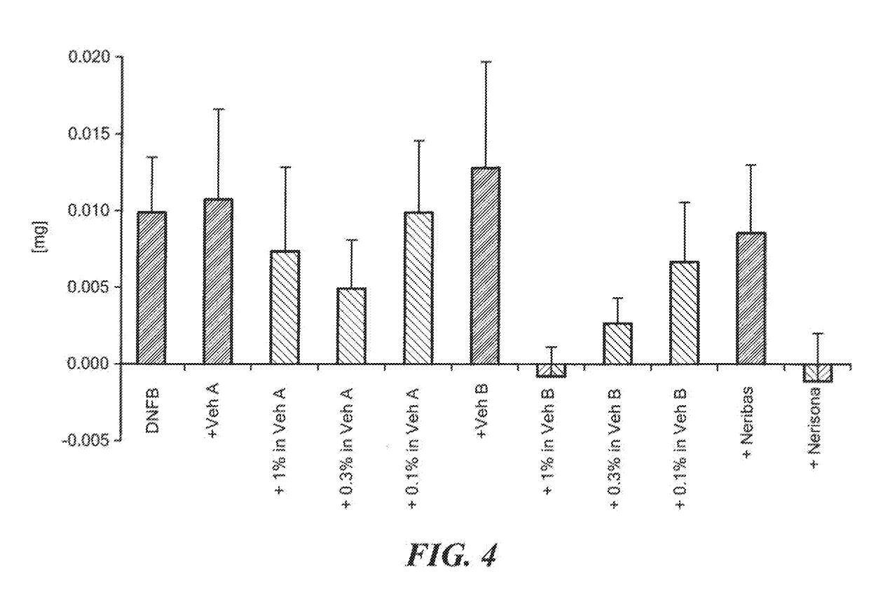

FIG. 4

Results of an induced contact dermatitis in mice (measured value: ear weight) according to Example 8

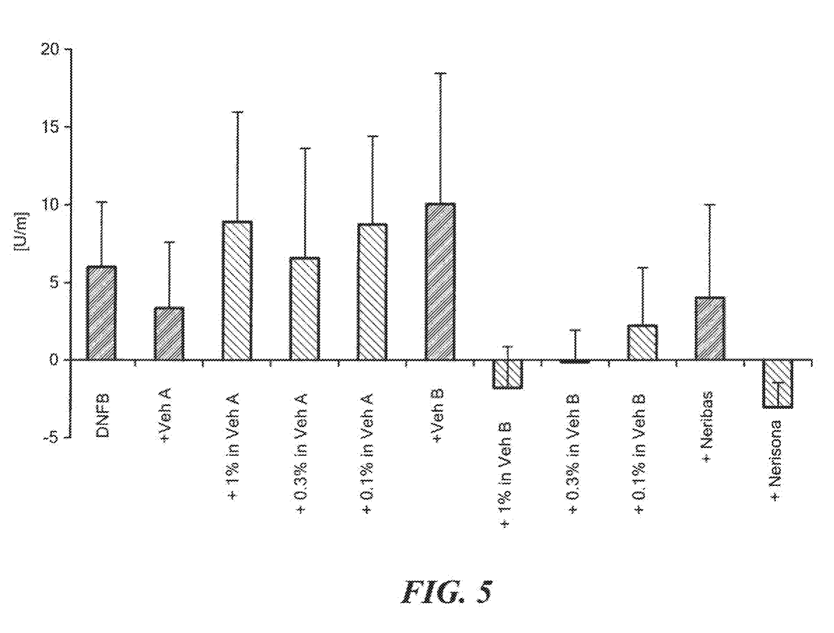

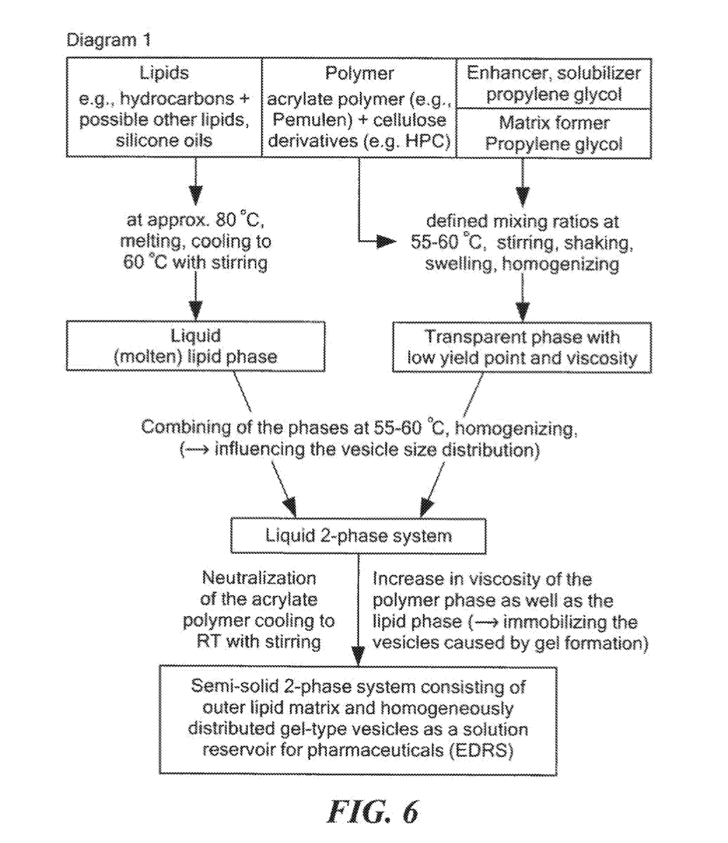

FIG. 5

Results of an induced contact dermatitis in mice (measured value: peroxidase) according to Example 8

The following examples explain the invention, however without limiting it. It is expressly claimed that advantageous effects are presented in the following examples only for illustration, and we do not wish to present these as a conclusive listing.

### EXAMPLES 1 TO 5

The following Table 1 shows exemplary formulations (ad 100 g) for the anhydrous multiphase gel system according to the invention, based on semi-solid lipid formulations with gelled mixtures of propylene carbonate and propylene glycol or ethanol. Table 1 shows compositions according to the invention based on Examples 1 to 5. The first column shows the components used in the production. The second column indicates the limits for the amount (in wt. %) of the respective components that can be present in the finished formulation. Columns three to seven give selected formulations (quantity data in wt. % each time). In Examples 3 and 4 (columns five and six), the compositions according to the invention contain ascrolimus as the active substance.

Table 1 thus shows that the quantities of individual components for obtaining the composition according to the invention with advantageous properties can vary within broad limits.

It is thus possible for the person skilled in the art, without anything further, to determine with the help of a few tests, a formulation which dissolves even very difficultly soluble active substances in the composition according to the invention, in order to assure their application onto or throughout the skin. An example of an active substance that almost cannot be topically applied with topical preparations according to the prior art is presented in Examples 3 and 4. The topical macrolide ascrolimus has been introduced here in a high concentration (up to 1.0 wt. %) in a composition according to the invention.

TABLE 1

| Component | Limits | Example 1 | Example 2 | Example 3 | Example 4 | Example 5 |
|---|---|---|---|---|---|---|
| Ascrolimus (active substance) | 0-1.0 | — | — | 1.0 | 0.1 | — |
| Carbomer copolymer | 0.1-0.6 | 0.45 | 0.3 | 0.15 | 0.15 | 0.40 |
| (Pemulen TR-1/TR-2) | | | | | | |
| Carbomer (Carbopol 980) | 0-0.3 | 0.17 | — | — | — | 0.17 |
| Hydroxypropylcellulose | 0-0.4 | 0.12 | — | — | — | — |
| (Klucel HF) | | | | | | |
| Hydroxyethylmethylcellulose | 0-0.4 | — | 0.15 | — | — | — |
| (Tylopur MH 1000) | | | | | | |
| Propylene carbonate | 5.0-15.0 | 14.75 | 9.775 | 5.0 | 5.0 | — |
| Ethanol 96% | 0-20 | — | — | — | — | 19.0 |
| Propylene glycol | 5.0-15.0 | 14.75 | 9.775 | 5.0 | 5.0 | |
| Diisopropylamine | 0-0.1 | 0.075 | 0.024 | — | — | 0.05 |
| White vaseline | 40.0-80.0 | 47.685 | 57.976 | 65.85 | 66.75 | 59.95 |
| Paraffin oil, viscous | 0-10.0 | 10.0 | 10.0 | 10.0 | 10.0 | 10.0 |
| Cyclomethicone | 0-10.0 | 3.0 | — | — | — | 3.0 |
| Beeswax (Cera Alba) | 0-5.0 | 2.0 | 5.0 | 5.0 | 5.0 | 2.0 |
| Hard paraffin | 0-7.0 | 7.0 | 7.0 | 5.0 | 5.0 | 7.0 |
| Microcrystalline wax | 0-5.0 | — | — | 3.0 | 3.0 | — |

### EXAMPLE 6

### General Preparation Technology for an EDRS

Diagram 1 illustrated below shows the general preparation technology for the EDR system according to the invention. Depending on the desired objective each time, the active substances to be introduced into the system are introduced either into the lipid phase or into the polymer phase, or even into both phases. The temperatures at which the individual production steps take place can exercise an influence. The lipids must almost always be melted, while the polymer phase is already sufficiently liquid under special conditions of the composition, so that another liquefying, for example, by heating can be omitted. When the phases are combined, the vesicle size distribution can then be influenced as desired. With subsequent cooling of the mixtures, the vesicles are then immobilized with the formation of a gel. The individual preparation steps are thoroughly familiar to the person skilled in the art, a pharmacist. Varying the appropriate parameters in order to obtain the optimal EDRS is thus possible and easy for the person skilled in the art, without anything further. The preparation diagram given here is not to be understood as a limitation. Common method variations are viewed as belonging to the scope of the invention.

### EXAMPLE 7

### Study of Application on Human Skin

An investigation of the compatibility of the composition according to the invention in comparison to two formulations according to the prior art is described below. The compositions for topical treatment of atopic dermatitis were utilized for a period of one week on an individual target lesion. The test series was conducted as a double-blind, randomized, single-center, two-period crossover investigation.

18 test subjects (female and male, Caucasian, aged 19 to 41 with at least two lesions of 4 cm² in size) participated in the study up to the end; a statistical distribution was not found. The test subjects received two different treatments on different lesions each time within two successive weeks. Only individual applications by individual test subjects were omitted.

Three Preparations were Applied:
Vehicle A: Lipid salve (prior art)
Vehicle B: Anhydrous multiphase gel system (EDRS) according to the invention
Vehicle C: Water-in-oil preparation (prior art)

### Measured Values:

1. The test subjects had to evaluate, on a questionnaire, the feeling on the skin, including the occurrence of itching, wherein the value range was 0 to 100.
2. The test subjects had to indicate the overall impression of the respective treatments.
3. Comments of any type from the test subjects, expressed spontaneously or upon inquiry, were collected (everything that displeased the test subject, any perceived sensation; skin irritations, pimples/rashes, warming of the skin, cold sensation, stinging, tingling, burning, itching, skin swelling, pain, etc.).
4. Evaluation of the overall severity of the lesions was assessed by a physician (assessment of the target lesions by the physician: The severity of the erythema, edema/papulation, discharge/encrustation, excoriation, lichenification were assessed and evaluated on a 4-point scale).
5. Photo documentation, negative incident report, blood pressure, heart rate were measured or prepared.

### Results:

For 1. The results are shown in FIG. 1. The mean value (bars) and the standard deviation (line) are plotted in mm on the x-axis.

The test subjects were questioned according to the following parameters: Overall impression (GE), penetration into the skin (EH), stickiness (KL), greasiness (FE), itchiness during treatment (JB), itchiness during application (JA), skin dryness (HT), moistness (FT), odor (GR).

It is clear that the composition according to the invention (vehicle B) in most cases is comparable to the water-in-oil formulation (vehicle C), which is generally experienced as particularly pleasant on the skin. It is particularly noteworthy that itching, both directly during application as well as in the course of treatment, was assessed as significantly less in comparison to the other vehicles A and C.

For 2. Seven test subjects overall preferred vehicle C, five test subjects preferred vehicle A and likewise five test subjects preferred vehicle B (EDRS according to the invention).

For 3. The test subjects did not provide spontaneous comments. On the questionnaires, the comments of the test subjects were concentrated in three areas, namely skin irritation, pimples/rash, and itching. The results for the measured values are shown graphically in FIGS. 2a to 2c: skin irritation (FIG. 2a), pimples/rash (FIG. 2b) and itching (FIG. 2c). The scope of evaluation comprises a scale (y-axis) from 0 to 12 and considers the intensity of the experience (0-3) and its duration (1-4). Two bars are plotted each time on the x-axis for vehicle A (A), vehicle B (B) and vehicle C (C), wherein the left column each time indicates the experience before treatment and the right column each time indicates the experience after treatment with the respective vehicle. It is shown that the EDRS according to the invention (vehicle B) in all cases produced significant results. The treatment effect is particularly clear in the case of skin irritations and with itching.

For 4. The evaluation of the overall severity of the lesions is shown in FIG. 3. Two bars are plotted each time on the x-axis for vehicle A (A), vehicle B (B) and vehicle C (C). The left column of the pair of columns each time shows the severity prior to the respective treatment. The right column each time gives the severity of the lesions after the treatment with the respective vehicle. It is clearly shown that vehicle B (EDRS according to the invention) significantly showed the best effect.

For 5. During the study, three negative incidents were reported:

Test subject 1: Bronchitis, which was not related to the treatment under study
Test subject 2: Urticaria of mild intensity, far removed from the lesions, observed earlier in the subject
Test subject 8: Exacerbation of atopic dermatitis in the treated lesion, probably caused by vehicle C.

In summary, it is established that the classical water-in-oil formulation is generally preferred by the test subjects. Of course, the EDRS according to the invention (vehicle B) has considerable advantages in comparison to the formulations of the prior art, particularly in the case of therapeutically important properties, such as itching, skin irritations and in the overall effect.

### EXAMPLE 8

### DBFB-Induced Contact Dermatitis in Mice

Various models can be used in order to determine the effectiveness and bioavailability of active substances as a function of the topical medication used. A suitable pharmacological model within the scope of anti-inflammatory therapy on the skin is represented by the “Irritant contact dermatitis (ICD)” model in mice. In this model, the inflammatory components of eczema are imitated. This inflammatory aspect likewise plays a role in allergic contact dermatitis and atopic dermatitis with the participation of allergen-specific T cells intrinsic to the skin. Therefore, a T-cell-dependent model of allergic contact dermatitis (in reaction to dinitrofluorobenzene, DNFB) can be utilized.

Acute and chronic allergic contact dermatitis (ACD) is characterized by a type-1 dominated cytokine profile. Therefore, the ACD model induced by dinitrofluorobenzene (DNFB) was used in order to assess in vivo the activity of the active substance “ascrolimus” (topical immunomodulator). After sensitizing by means of DNFB, edema was produced by subsequent application of the same contact allergens and therefore, a cutaneous infiltration of allergenic T-cells and granulocytes was triggered.

The mice (NMRI) were sensitized with 0.5% DNFB on day 0 and day 1. On day 5, the mice were again treated with 0.3% DNFB. The test products containing the active substance ascrolimus were co-applied topically in three different concentrations (0.1, 0.3, 1.0%) in a standard lipid salve as well as with the EDRS according to the invention. After 24 hours, the animals were sacrificed in order to measure ear weight, elastase and peroxidase activity of ear homogenates as parameters for edema and granulocyte infiltration. Each experiment was repeated twice.

The results of the measurement of ear weight as a measure of edema formation show for the EDRS-verum salve a clear, concentration-dependent reduction of the induced inflammatory effects in comparison to the standard lipid salve. No significant reduction in the ear weight (y-axis=delta ear weight in mg [mg]) is shown here in comparison to the placebo in the case of the same 1% concentration of ascrolimus (FIG. 4).

The peroxidase activity as a measure for granulocyte infiltration (FIG. 5) likewise showed a significant concentration-dependent inhibition when ascrolimus was applied in the EDR system (y-axis=delta peroxidase in units per ml [U/nml]). In comparison, a significant effect could not be measured with the standard lipid salve. Only the internal standard (glucocorticoid) also showed an inhibition of edema formation and granulocyte infiltration.

### Legend to FIGS. 4 and 5:

Vehicle A=Standard lipid salve (placebo)
Vehicle B=EDRS (placebo)
0.1, 0.3, 1.0% in vehicle A or B=concentrations of ascrolimus in the vehicle
Neribas=base (placebo); internal standard
Nerisona=standard lipid salve containing diflucortolone pivalate (glucocorticoid); internal standard

It could be shown, for example, that the systems according to the invention (EDRS) are sufficiently stable and can be well tolerated. In type and quantity, the solvent mixture in the hydrocarbon system can be varied within certain limits as a function of the physico-chemical properties and the potency of drugs.

1. An anhydrous multiphase gel system consisting of an outer lipid matrix and an inner phase gelled by means of a polymer, characterized in that the lipid phase contains lipids that are compatible with the skin and that the lipid phase is semi-solid or solid at room temperature, in that the polymers involve cellulose derivatives, acrylate polymers and their derivatives or their mixture, in that the polymers or polymer blends that can be swollen are swollen by means of swelling agents containing OH groups, and in that the swelling agent additionally comprises carbonic acid diesters or mixtures of carbonic acid diesters, that is obtained by
a) Melting the lipid phase with the formation of a liquid lipid phase,
b) Mixing and homogenizing polymers or polymer blends capable of swelling with the formation of a polymer phase to be dispersed,
c) Combining the polymer phase with the liquid lipid phase and homogenizing the phases, and
d) Cold stirring the phase mixture at room temperature until a solid gel mixed structure of the entire system is formed.

2. (canceled)

3. The system according to claim 1, further characterized in that the lipids are selected from petrolatum, paraffin, microcrystalline wax, squalene, cetylstearyl octanoate, ethyl oleate, myristyl myristate, propylene glycol dicaprate, cetyl esters, isopropyl myristate, isopropyl palmitate, mono-, di- and triglycerides, ethoxylated glycerides, polyethylene glycol esters, sorbitan esters, solid lipids, dibutyl adipate, ethyl linoleate, propylene glycol isoceteth-3 acetate, ethylhexyl cocoate, isocetyl stearate, oleyloleate, cetyl palmitate, cetyl alcohol, oleyl alcohol, stearyl alcohol, dicaprylyl ether, oleic acid, waxes, cholesterins, polyethylene glycols, lanolin, lanolin alcohols, silicone oils and their mixtures, in that the cellulose derivatives involve hydroxypropylcellulose, carboxymethylcellulose, methylcellulose, hydroxypropylmethylcellulose, hydroxyethylcellulose and their derivatives or their mixture, in that the acrylate polymers involve crosslinked acylate polymers, and in that the swelling agent is a monohydric to trihydric aliphatic alcohol with a chain length of Up to 5 carbon atoms or mixtures thereof.

4. (canceled)

5. (canceled)

6. (canceled)

7. (canceled)

8. (canceled)

9. The system according to claim 1, further characterized in that the carbonic acid diesters are selected from the group: ethylene carbonate, propylene carbonate and other homologs of ethylene carbonate and mixtures thereof.

10. The system according to claim 1, further characterized in that the swelling agent additionally comprises diethylene glycol monoethyl ether, polyoxylated capryl/capric acid glycerides, dimethyl isosorbide and/or additional pharmaceutically compatible solvents or mixtures thereof.

11. The system according to claim 1, further characterized in that the lipid phase and/or the polymer phase additionally contain active substances, which may be different active substances.

12. (canceled)

13. (canceled)

14. The system according to claim 11, further characterized in that the active substances are selected from polidocanol, synthetic tanning substances, antiseptics, antibiotics, antimycotics, topical corticosteroids, topical macrolides, oligonucleotides for gene therapy, antihistamines, immunosuppressants, dithranol, vitamin D3 analogs, topical retinoids, urea, lactic acid, fumaric acid ester, azelaic acid, hydroquinone, benzoyl peroxide, non-steroidal antiphlogistics, sex hormones, cytostatics, UV protectors, plant extracts, and fruit acids.

15. The system according to claim 11, further characterized in that the active substance is introduced together with a solubilizing agent.

16. (canceled)

17. (canceled)

18. (canceled)

19. The system according to claim 1, further characterized in that the combining step c) is performed at a temperature of 55-60° C.

20. (canceled)

21. (canceled)

22. (canceled)

23. The system according to claim 14, further characterized in that sex hormone is selected from the group consisting of estrogens and androgens.

24. The system according to claim 14, further characterized in that the UV protector is stilbene derivative.

25. The system according to claim 14, further characterized in that the vitamin D3 analog is calcipotriol.

26. The system according to claim 14, further characterized in that the fruit acid is selected from the group consisting of α-hydroxy acids, β-hydroxy acids, polyhydroxy acids, malic acid, malonic acid, citric acid, or mixtures thereof.

27. The system according to claim 14, further characterized in that the antiseptic is selected from the group consisting of chlorhexidine and triclosan.

28. The system according to claim 14, further characterized in that the antibiotic is selected from the group consisting of fusidic acid, erythromycin, tetracycline, clindamycin, and peptide antibiotics.

29. The system according to claim 14, further characterized in that the antimycotic is selected from the group consisting of imidazole derivatives, terbenafine, ciclopirox, salicylic acid, and zinc pyrithione.

30. The system according to claim 14, further characterized in that the oligonucleotide for gene therapy is selected from the group consisting of si-RNA and ribozymes.

31. The system according to claim 14, further characterized in that the immunosuppressant is selected from the group consisting of cyclosporine, azathioprine, and mycophenolate mofetil.

32. The system according to claim 14, further characterized in that the topical corticosteroid is selected from the group consisting of methylprednisolone aceponate, clobetasol, and mometasone fuorate.

33. The system according to claim 14, further characterized in that the topical macrolide is selected from the group consisting of ascrolimus, tacrolimus and pimecrolimus.

34. The system according to claim 14, further characterized in that the plant extract is selected from the group consisting of green tea extract, Centella asiatica extract, willow bark extract, birch extract, tea olive oil, olive leaf extract, Aloe vera extract, marigold extract, passion flower extract, Hamamelis extract, chamomile extract, bearberry leaf extract and licorice root extract as 18β-glycyrhetinic acid Zn combination.

35. (canceled)

36. (canceled)

37. (canceled)
